(19) United States
(12) Patent Application Publication
(10) Pub. No.: US 2016/0166730 A1
(43) Pub. Date: Jun. 16, 2016

(54) Biocompatable Annular Prostheses and Methods for Forming Same

(71) Applicant: CorMatrix Cardiovascular, Inc., Roswell, GA (US)

(72) Inventor: Robert G. Matheny, Norcross, GA (US)

(21) Appl. No.: 14/953,548

(22) Filed: Nov. 30, 2015

Related U.S. Application Data
(60) Provisional application No. 62/090,256, filed on Dec. 10, 2014.

(51) Int. Cl. A61L 27/36; A61L 27/18; A61F 2/24; A61L 27/54

(52) U.S. Cl. CPC A61L 27/3633; A61L 27/54; A61L 27/18; A61F 2/2445; A61L 2300/41; A61L 2300/414; A61L 2430/20; A61F 2210/0004; A61F 2230/0069

(57) ABSTRACT

Annular prostheses comprising an elongated tubular member formed from an extracellular matrix (ECM) composition comprising ECM from a mammalian tissue source, which, when disposed proximate damaged cardiovascular tissue, induces modulated healing, including modulation of inflammation and bioremodeling. The ECM can also be augmented with a supplemental biologically active agent, such as a growth factor, to enhance modulation of inflammation and bioremodeling.

### CROSS-REFERENCE TO RELATED PATENT APPLICATIONS

This application claims the benefit of U.S. Provisional Application No. 62/090,256, filed on Dec. 10, 2014.

### FIELD OF THE INVENTION

The present invention generally relates to prostheses for replacing defective cardiovascular valve annuli. More particularly, the present invention relates to bioremodelable annular prostheses for replacing defective aortic, pulmonary, mitral and tricuspid annuli and methods for forming same.

### BACKGROUND OF THE INVENTION

As is well known in the art, the human heart has four valves that control blood flow circulating through the human body. On the left side of the heart is the mitral valve, located between the left atrium and the left ventricle, and the aortic valve, located between the left ventricle and the aorta. Both of these valves direct oxygenated blood from the lungs into the aorta for distribution through the body.

The tricuspid valve, located between the right atrium and the right ventricle, and the pulmonary valve, located between the right ventricle and the pulmonary artery, however, are situated on the right side of the heart and direct deoxygenated blood from the body to the lungs.

The valve structure of the human heart is organized via the fibrous skeleton of the heart, which is a high density single structure of connective tissue that forms and anchors the valves and influences the forces exerted through them. The connective tissue is formed primarily of collagen and is electrically impermeable, thus, providing an electrical barrier between the atria and the ventricles. The skeleton also anchors the valves, providing a support structure referred to as the valve annulus.

The peripheral venous system also includes a number of valves that prevent retrograde blood flow. By preventing retrograde blood flow, the valves found throughout the venous system assist the flow of blood through the veins and returning to the heart.

Normally, the mitral valve has two leaflets and the tricuspid valve has at least two, preferably three leaflets. The aortic and pulmonary valves, however, have normally at least two, preferably three leaflets, also often referred to as “cusps” because of their half-moon like appearance.

Venous valves are usually of the bicuspid type, with each cusp or leaflet forming a reservoir for blood, which, under pressure, forces the free edges of the cusps together to permit mostly antegrade blood flow to the heart. As discussed in detail below, since a majority of venous blood flow is against gravity while a person is standing, incompetent or destroyed venous valves can cause significant medical problems in the legs, ankles, and feet.

Valve diseases are typically classified into two major categories; stenosis and insufficiency. In the case of a stenosis, the native valve does not open properly, whereby insufficiency represents the opposite effect showing deficient closing properties.

Insufficiency of the inlet (atrioventricular) tricuspid valve to the right ventricle of the heart results in regurgitation of blood back into the right atrium, which. serving to receive blood flow returning in the veins from the entire body, then results in turn in suffusion and swelling (edema) of all the organs, most notably in the abdomen and extremities, insufficient forward conduction of blood flow from the right ventricle into the lungs causing compromise of pulmonary function, and ultimately pump failure of the right heart. Collectively these conditions are termed right heart failure, a condition that leads to incapacity and possibly to death if progressive and uncorrected.

One common problem associated with valve diseases is an enlargement of the valve annulus. The annulus may enlarge or dilate to a point where the attached leaflets are unable to fully close, which may lead to regurgitation. As a result, surgical correction, either by valve repair procedures or by valve replacement, may be required.

Surgical reconstruction can include remodeling of the valve annulus (e.g., annuloplasty), which may be accomplished by implantation of a prosthetic annuloplasty ring to help stabilize the annulus and to correct or prevent the valvular insufficiency caused by the annular defect. By properly sizing and implanting the annuloplasty ring, the valve annulus can be substantially restored to its normal, undilated, circumference.

Various polymer based apparatus have been developed in an attempt to remodel a defective and/or failed valve annulus. Illustrative are the polymer based apparatus, i.e. annuloplasty rings, disclosed in U.S. Pat. Nos. 8,574,289, 8,236,051, 7,887,583 and U.S. application Ser. No. 10/677,104. Other commercially available annuloplasty rings are rigid or flexible plastic material covered with a biocompatible cloth such as a Dacron® (Polyethylene terephthalate).

A major drawback of the noted polymer based apparatus, as well as most known apparatus, is that the apparatus often comprise or include a permanent structure that remains in the body, i.e. non-biodegradable. As is well known in the art, such structures (or devices) can, and in most instances will, cause irritation and undesirable biologic responses in the surrounding tissue, such as embolism at the annulus and other thromboembolic events in the region necessitating medical attention.

Annular implants have also been made of smooth animal pericardium taken from horses, cows and pigs that has been fixed. The pericardium is crosslinked with glutaraldehyde and undergoes a detoxification process with heparin bonding, such as one of the BioRing® natural tissue products that are commercially available from Shelhigh, Inc. of Millburn, N.J.

A major disadvantage of crosslinking pericardium is, however, that it reduces the antigenicity of the material by linking the antigenic epitopes, rendering them either inaccessible to phagocytosis or foreign to the immune system.

Crosslinking pericardium will thus, in general, generate collagenous material that resembles a synthetic material more than a natural biological tissue, both mechanically and biologically.

As is well known in the art, crosslinked prosthetic tissue implants; more particularly, glutaraldehyde crosslinked tissue, often undergo late calcific degeneration when implanted into a mammalian heart, which results in prosthesis failure. Deposits from the calcific degeneration process substantially increase the risk for thromboembolic and/or calcium-related embolic events.

There is thus a need to provide biocompatible annular prostheses that can be readily employed to selectively replace diseased or defective aortic, pulmonary, mitral, and tricuspid annuli.

It is therefore an object of the present invention to provide biocompatible annular prostheses that can be readily employed to selectively replace diseased or defective aortic, pulmonary, mitral, and tricuspid annuli.

It is another object of the present invention to provide a method for forming biocompatible annular prostheses that can be readily employed to selectively replace diseased or defective aortic, pulmonary, mitral, and tricuspid annuli.

It is another object of the present invention to provide biocompatible annular prostheses that substantially reduce or eliminate calcific degeneration after intervention in a valve and the harsh biological responses associated with conventional polymeric and metal valves.

It is another object of the present invention to provide extracellular matrix (ECM) annular prostheses that induce host tissue and/or cell proliferation, bioremodeling and regeneration of new tissue and tissue structures with site-specific structural and functional properties.

It is another object of the present invention to provide extracellular matrix (ECM) annular prostheses that are capable of administering a pharmacological agent to host tissue and, thereby produce a desired biological and/or therapeutic effect.

As will readily be appreciated by one having ordinary skill in the art, the annular prostheses of the invention provide numerous advantages over conventional apparatus for repairing and/or regenerating tissue. Among the advantages are the following:
The provision of annular prostheses that substantially reduce or eliminate (i) the harsh biological responses associated with conventional polymeric and metal ECM based and non-ECM apparatus, and (ii) the formation of inflammation and infection after deployment;
The provision of annular prostheses that can be readily and effectively employed to treat damaged or diseased biological tissue; particularly, cardiovascular tissue;
The provision of annular prostheses that induce host tissue and/or cell proliferation, bioremodeling and regeneration of new tissue, and tissue structures with site-specific structural and functional properties; and
The provision of annular prostheses that effectively administer at least one biologically active agent and/or pharmacological agent or composition to a subject's tissue and, thereby produce a desired biological and/or therapeutic effect.

### SUMMARY OF THE INVENTION

The present invention is directed to biocompatible annular prostheses and methods for forming same.

As indicated above, in a preferred embodiment, the annular prostheses comprise at least one elongated tubular member.

In some embodiments, the tubular member comprises a rolled biocompatible sheet.

In some embodiments, the tubular member comprises a plurality of laminate sheets.

In some embodiments, the tubular member comprises a plurality of biocompatible strands.

According to the invention, the annular prostheses are formed by orienting the tubular member in a substantially circular and/or elliptical configuration and securing the first end to the second end at a junction.

In some embodiments of the invention, the annular prostheses comprise ECM material derived from a mammalian tissue source selected from the group comprising, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ. The ECM can also comprise collagen from mammalian sources.

In a preferred embodiment, the mammalian ECM material referenced above comprises sterilized acellular ECM material.

Preferably, the mammalian tissue sources referenced above comprise an adolescent mammalian tissue source.

In some embodiments of the invention, the annular prosthesis comprises a biocompatible polymeric composition.

According to the invention, the polymeric composition can comprise, without limitation, polycaprolactone (PCL), Artelon® (porous polyurethaneurea), polyglycolide (PGA), polylactide (PLA), poly(ε-caprolactone) (PCL), poly dioxanone (a polyether-ester), poly lactide-co-glycolide, polyamide esters, polyalkalene esters, polyvinyl esters, polyvinyl alcohol, and polyanhydrides. Natural polymeric compositions, include, without limitation, polysaccharides (e.g. starch and cellulose), proteins (e.g., gelatin, casein, silk, wool, etc.), and polyesters (e.g., polyhydroxyalkanoates).

The polymeric composition can also comprise a hydrogel composition, including, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide.

In some embodiments of the invention, the annular prostheses comprise an ECM-mimicking biomaterial composition.

In some embodiments, the ECM-mimicking biomaterial composition comprises poly(glycerol sebacate) (PGS).

In some embodiments of the invention, the annular prostheses comprise at least one additional biologically active agent, i.e. an agent that induces or modulates a physiological or biological process, or cellular activity, e.g., induces proliferation, and/or growth and/or regeneration of cells and/or tissue.

In some embodiments, the biologically active agent comprises a cell selected from the group comprising, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells.

In some embodiments, the biologically active agent comprises a growth factor selected from the group comprising, without limitation, transforming growth factor alpha (TGF-α), transforming growth factor beta (TGF-β), fibroblast growth factor-2 (FGF-2), basic fibroblast growth factor (bFGF), vascular epithelial growth factor (VEGF), and insulin-like growth factor (IGF).

In some embodiments, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments of the invention, the annular prostheses comprise at least one pharmacological agent or composition, i.e. an agent, drug, compound, composition of matter or mixture thereof, including its formulation, which provides some therapeutic, often beneficial, effect.

In a preferred embodiment, the pharmacological agent or composition is selected from the group comprising, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or anti-thrombic agents, DNA, RNA, modified DNA and RNA, NSAIDs, and inhibitors of DNA, RNA or protein synthesis.

In some embodiments of the invention, the annular prostheses provide a single-stage agent delivery profile, i.e. comprise a single-stage agent delivery vehicle, wherein a modulated dosage of an aforementioned biologically active and/or pharmacological agent is provided.

In some embodiments of the invention, the annular prostheses provide a multi-stage agent delivery profile, i.e. comprise a multi-stage delivery vehicle, wherein a plurality of the aforementioned biologically active and/or pharmacological agents are administered via a modulated dosage.

In some embodiments of the invention, the annular prostheses further comprise an outer reinforcing structure.

In some embodiments of the invention, the reinforcing structure comprises a thin member, such as a strand, that is wound about the outer surface of the annular prosthesis.

In some embodiments of the invention, the reinforcing structure comprises a mesh or woven structure.

In some embodiments of the invention, the reinforcing structure comprises an ECM-mimicking biomaterial, such as PGS.

In another embodiment of the invention, there is provided a method of temporarily positioning and anchoring the annular prostheses of the invention.

In another embodiment of the invention, there is provided a method of forming the aforementioned annular prostheses of the invention.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a top plan view of an embodiment of the annular prosthesis, in accordance with the invention;

FIGS. 2-4 are perspective sectional views of three embodiments of annular prostheses shown in FIG. 1, in accordance with the invention;

FIG. 5 is a perspective view of laminate sheets, in accordance with the invention.

FIG. 6A is a perspective sectional view of rolled laminate sheets forming a tubular member, in accordance with the invention;

FIG. 6B is a perspective sectional view of a tubular member shown in FIG. 3A, in accordance with the invention;

FIG. 7A is a perspective sectional view of rolled laminate sheets having bound first and second ends forming a tubular member, in accordance with the invention, in accordance with the invention;

FIG. 7B is a perspective sectional view of a tubular member shown in FIG. 4A, in accordance with the invention;

FIG. 8 is a perspective sectional view of a bicuspid valve structure having an embodiment of the annular prosthesis of FIG. 2, in accordance with the invention;

FIG. 9 is a perspective sectional view of a tricuspid valve structure having an embodiment of the annular prosthesis of FIG. 2, in accordance with the invention;

FIG. 10 is a graphical illustration reflecting the effect of a statin augmented ECM on MCP-1 mRNA expression between two time points, in accordance with the invention;

FIG. 11 is a graphical illustration reflecting the effect of a statin augmented ECM on CCR2 mRNA expression between two time points, in accordance with the invention;

FIG. 12 is a graphical illustration reflecting the effect of a statin augmented ECM on RAC 1 mRNA expression between two time points, in accordance with the invention; and

FIG. 13 is a graphical illustration reflecting the effect of a statin augmented ECM on MCP-1 concentration and mRNA expression over the course of five time points, in accordance with the invention.

### DETAILED DESCRIPTION OF THE PREFERRED EMBODIMENT

Before describing the present invention in detail, it is to be understood that this invention is not limited to particularly exemplified apparatus, systems, materials, compositions, structures or methods as such may, of course, vary. Thus, although a number of apparatus, systems, materials, compositions, structures and methods similar or equivalent to those described herein can be used in the practice of the present invention, the preferred apparatus, systems, materials, compositions, structures and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

As used in this specification and the appended claims, the singular forms “a, “an” and “the” include plural referents unless the content clearly dictates otherwise. Thus, for example, reference to “an active” includes two or more such actives and the like.

Further, ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

It is also understood that there are a number of values disclosed herein, and that each value is also herein disclosed as “approximately” that particular value in addition to the value itself. For example, if the value “10” is disclosed, then “approximately 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed then “less than or equal to 10”, as well as “greater than or equal to 10” is also disclosed.

### DEFINITIONS

The term “prosthesis” as used herein, and means and includes a device member or system that is configured for placement on biological tissue on or in an organ, such as a lumen or vessel. As discussed in detail herein, upon placement of a graft of the invention to biological tissue; particularly, damaged or diseased tissue the graft induces “modulated healing”.

The term “woven”, as used herein, means and includes an ordered arrangement of fibers bonded by physical, mechanical, or chemical means.

The term “nonwoven”, as used herein, refers to an arrangement of fibers bonded by random and/or semi-random entanglement, and/or physical, mechanical or chemical means as opposed to weave or knitted fabrics where the structure is highly ordered. The orientation of the fibers in a nonwoven can be either random or have some degree of order.

The term “biocompatible”, as used herein, means a device or material that is substantially non-toxic in an in vivo environment, and is not substantially rejected by a recipient's physiological system, i.e. non-antigenic.

The terms “extracellular matrix” and “ECM” are used interchangeably herein, and mean and include a collagen-rich substance that is found in between cells in mammalian tissue, and any material processed therefrom, e.g. decellularized ECM. According to the invention, the ECM material can be derived from various mammalian tissue sources including, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The ECM material can thus comprise, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, omentum extracellular matrix, epithelium of mesodermal origin, i.e. mesothelial tissue, cardiac extracellular matrix, e.g., pericardium and/or myocardium, kidney extracellular matrix, pancreas extracellular matrix, lung extracellular matrix, and combinations thereof. The ECM can also comprise collagen from mammalian sources.

The terms “urinary bladder submucosa (UBS)”, “small intestine submucosa (SIS)” and “stomach submucosa (SS)” also mean and include any UBS and/or SIS and/or SS material that includes the tunica mucosa (which includes the transitional epithelial layer and the tunica propria), submucosal layer, one or more layers of muscularis, and adventitia (a loose connective tissue layer) associated therewith.

The ECM can also be derived from basement membrane of mammalian tissue/organs, including, without limitation, bladder, “urinary basement membrane (UBM)”, liver, i.e. “liver basement membrane (LBM)”, and amnion, chorion, allograft pericardium, allograft acellular dermis, amniotic membrane, Wharton's jelly, and combinations thereof.

Additional sources of mammalian basement membrane include, without limitation, spleen, lymph nodes, salivary glands, prostate, pancreas and other secreting glands.

The ECM can also be derived from other sources, including, without limitation, collagen from plant sources and synthesized extracellular matrices, i.e. cell cultures.

The term “angiogenesis”, as used herein, means a physiologic process involving the growth of new blood vessels from pre-existing blood vessels.

The term “neovascularization”, as used herein, means and includes the formation of functional vascular networks that can be perfused by blood or blood components. Neovascularization includes angiogenesis, budding angiogenesis, intussuceptive angiogenesis, sprouting angiogenesis, therapeutic angiogenesis and vasculogenesis.

The terms “biologically active agent” and “biologically active composition” are used interchangeably herein, and mean and include agent that induces or modulates a physiological or biological process, or cellular activity, e.g., induces proliferation, and/or growth and/or regeneration of cells and/or tissue.

The terms “ECM-mimicking biomaterial”, and “ECM-mimicking material” are used interchangeably herein, and mean and include a biocompatible and biodegradable biomaterial that induces neovascularization and bioremodeling of tissue in vivo, i.e. when disposed proximate damaged biological tissue. The term “ECM-mimicking” thus includes, without limitation, ECM-mimicking biomaterial compositions; specifically, poly(glycerol sebacate) (PGS).

The terms “biologically active agent” and “biologically active composition” thus mean and include, without limitation, the following growth factors: platelet derived growth factor (PDGF), epidermal growth factor (EGF), transforming growth factor alpha (TGF-α), transforming growth factor beta (TGF-β), fibroblast growth factor-2 (FGF-2), basic fibroblast growth factor (bFGF), vascular epithelial growth factor (VEGF), hepatocyte growth factor (HGF), insulin-like growth factor (IGF), nerve growth factor (NGF), platelet derived growth factor (PDGF), tumor necrosis factor alpha (TNF-α), and placental growth factor (PLGF).

The terms “biologically active agent” and “biologically active composition” also mean and include, without limitation, embryonic stem cells, mesenchymal stem cells, hematopoietic stem cells, bone marrow stem cells, bone marrow-derived progenitor cells, myosatellite progenitor cells, totipotent stem cells, pluripotent stem cells, multipotent stem cells, oligopotent stem cells and unipotent stem cells. The group also comprises cardiomyocytes, myoblasts, monocytes, parenchymal cells, epithelial cells, endothelial cells, mesothelial cells, fibroblasts, osteoblasts, chondrocytes, exogenous cells, endogenous cells, macrophages, capillary endothelial cells, xenogenic cells, allogenic cells, and cells derived from any of the three germ layers including the endoderm, mesoderm and ectoderm.

The terms “biologically active agent” and “biologically active composition” also mean and include, without limitation, the following biologically active agents (referred to interchangeably herein as a “protein”, “peptide” and “polypeptide”): collagen (types I-V), proteoglycans, glycosaminoglycans (GAGs), glycoproteins, cytokines, cell-surface associated proteins, cell adhesion molecules (CAM), endothelial ligands, matrikines, cadherins, immuoglobins, fibril collagens, non-fibrillar collagens, basement membrane collagens, multiplexins, small-leucine rich proteoglycans, decorins, biglycans, fibromodulins, keratocans, lumicans, epiphycans, heparin sulfate proteoglycans, perlecans, agrins, testicans, syndecans, glypicans, serglycins, selectins, lecticans, aggrecans, versicans, neurocans, brevicans, cytoplasmic domain-44 (CD-44), macrophage stimulating factors, amyloid precursor proteins, heparins, chondroitin sulfate B (dermatan sulfate), chondroitin sulfate A, heparin sulfates, hyaluronic acids, fibronectins, tenascins, elastins, fibrillins, laminins, nidogen/enactins, fibulin I, fibulin II, integrins, transmembrane molecules, thrombospondins, ostepontins, and angiotensin converting enzymes (ACE).

The terms “pharmacological agent”, “active agent”, “drug” and “active agent formulation” are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect or effects in animals, including warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The terms “pharmacological agent”, “active agent”, “drug” and “active agent formulation” thus mean and include, without limitation, antibiotics, anti-arrhythmic agents, anti-viral agents, analgesics, steroidal anti-inflammatories, non-steroidal anti-inflammatories, anti-neoplastics, anti-spasmodics, modulators of cell-extracellular matrix interactions, proteins, hormones, growth factors, matrix metalloproteinases (MMPs), enzymes and enzyme inhibitors, anticoagulants and/or anti-thrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of cells and/or tissue, and vasodilating agents.

The terms “pharmacological agent”, “active agent”, “drug” and “active agent formulation” thus include, without limitation, atropine, tropicamide, dexamethasone, dexamethasone phosphate, betamethasone, betamethasone phosphate, prednisolone, triamcinolone, triamcinolone acetonide, fluocinolone acetonide, anecortave acetate, budesonide, cyclosporine, FK-506, rapamycin, ruboxistaurin, midostaurin, flurbiprofen, suprofen, ketoprofen, diclofenac, ketorolac, nepafenac, lidocaine, neomycin, polymyxin b, bacitracin, gramicidin, gentamicin, oyxtetracycline, ciprofloxacin, ofloxacin, tobramycin, amikacin, vancomycin, cefazolin, ticarcillin, chloramphenicol, miconazole, itraconazole, trifluridine, vidarabine, ganciclovir, acyclovir, cidofovir, ara-amp, foscarnet, idoxuridine, adefovir dipivoxil, methotrexate, carboplatin, phenylephrine, epinephrine, dipivefrin, timolol, 6-hydroxydopamine, betaxolol, pilocarpine, carbachol, physostigmine, demecarium, dorzolamide, brinzolamide, latanoprost, sodium hyaluronate, insulin, verteporfin, pegaptanib, ranibizumab, and other antibodies, antineoplastics, anti-VEGFs, ciliary neurotrophic factor, brain-derived neurotrophic factor, bFGF, Caspase-1 inhibitors, Caspase-3 inhibitors, α-Adrenoceptors agonists, NMDA antagonists, Glial cell line-derived neurotrophic factors (GDNF), pigment epithelium-derived factor (PEDF), and NT-3, NT-4, NGF, IGF-2.

The terms “pharmacological agent”, “active agent”, “drug” and “active agent formulation” further mean and include the following Class I-Class V anti-arrhythmic agents: (Class Ia) quinidine, procainamide and disopyramide; (Class Ib) lidocaine, phenytoin and mexiletine; (Class Ic) flecainide, propafenone and moricizine; (Class II) propranolol, esmolol, timolol, metoprolol and atenolol; (Class III) amiodarone, sotalol, ibutilide and dofetilide; (Class IV) verapamil and diltiazem) and (Class V) adenosine and digoxin.

The terms “pharmacological agent”, “active agent”, “drug” and “active agent formulation” further mean and include, without limitation, the following antibiotics: aminoglycosides, cephalosporins, chloramphenicol, clindamycin, erythromycins, fluoroquinolones, macrolides, azolides, metronidazole, penicillins, tetracyclines, trimethoprim-sulfamethoxazole and vancomycin.

The terms “pharmacological agent”, “active agent”, “drug” and “active agent formulation” further include, without limitation, the following steroids: andranes (e.g., testosterone), cholestanes, cholic acids, corticosteroids (e.g., dexamethasone), estraenes (e.g., estradiol) and pregnanes (e.g., progesterone).

The terms “pharmacological agent”, “active agent”, “drug” and “active agent formulation” can further include one or more classes of narcotic analgesics, including, without limitation, morphine, codeine, heroin, hydromorphone, levorphanol, meperidine, methadone, oxycodone, propoxyphene, fentanyl, methadone, naloxone, buprenorphine, butorphanol, nalbuphine and pentazocine.

The terms “pharmacological agent”, “active agent”, “drug” and “active agent formulation” can further include one or more classes of topical or local anesthetics, including, without limitation, esters, such as benzocaine, chloroprocaine, cocaine, cyclomethycaine, dimethocaine/larocaine, piperocaine, propoxycaine, procaine/novacaine, proparacaine, and tetracaine/amethocaine. Local anesthetics can also include, without limitation, amides, such as articaine, bupivacaine, cinchocaine/dibucaine, etidocaine, levobupivacaine, lidocaine/lignocaine, mepivacaine, prilocaine, ropivacaine, and trimecaine. Local anesthetics can further include combinations of the above from either amides or esters.

The terms “anti-inflammatory” and “anti-inflammatory agent” are also used interchangeably herein, and mean and include a “pharmacological agent” and/or “active agent formulation”, which, when a therapeutically effective amount is administered to a subject, prevents or treats bodily tissue inflammation i.e. the protective tissue response to injury or destruction of tissues, which serves to destroy, dilute, or wall off both the injurious agent and the injured tissues.

Anti-inflammatory agents thus include, without limitation, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, decanoate, deflazacort, delatestryl, depo-testosterone, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenarnic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, mesterolone, methandrostenolone, methenolone, methenolone acetate, methylprednisolone suleptanate, momiflumate, nabumetone, nandrolone, naproxen, naproxen sodium, naproxol, nimazone, olsalazine sodium, orgotein, orpanoxin, oxandrolane, oxaprozin, oxyphenbutazone, oxymetholone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, semetacin, stanozolol, sudoxicam, sulindac, suprofen, talmetacin, talniflumate, talosalate, tebufelone, tenidap, tenidap sodium, tenoxicam, tesicam, tesimide, testosterone, testosterone blends, tetrydamine, tiopinac, tixocortol pivalate, tolmetin, tolmetin sodium, triclonide, triflumidate, zidometacin, and zomepirac sodium.

The term “pharmacological composition”, as used herein, means and includes a composition comprising a “pharmacological agent” and/or a “biologically active agent” and/or any additional agent or component identified herein.

The term “ECM composition”, as used herein, means and includes a composition comprising at least one ECM.

The term “therapeutically effective”, as used herein, means that the amount of the “pharmacological composition” and/or “pharmacological agent” and/or “biologically active agent” administered is of sufficient quantity to ameliorate one or more causes, symptoms, or sequelae of a disease or disorder. Such amelioration only requires a reduction or alteration, not necessarily elimination, of the cause, symptom, or sequelae of a disease or disorder.

The terms “prevent” and “preventing” are used interchangeably herein, and mean and include reducing the frequency or severity of a disease or condition. The term does not require an absolute preclusion of the disease or condition. Rather, this term includes decreasing the chance for disease occurrence.

The terms “treat” and “treatment” are used interchangeably herein, and mean and include medical management of a patient with the intent to cure, ameliorate, stabilize, or prevent a disease, pathological condition, or disorder. The terms include “active treatment”, i.e. treatment directed specifically toward the improvement of a disease, pathological condition, or disorder, and “causal treatment”, i.e. treatment directed toward removal of the cause of the associated disease, pathological condition, or disorder.

The terms “treat” and “treatment” further include “palliative treatment”, i.e. treatment designed for the relief of symptoms rather than the curing of the disease, pathological condition, or disorder, “preventative treatment”, i.e. treatment directed to minimizing or partially or completely inhibiting the development of the associated disease, pathological condition, or disorder, and “supportive treatment”, i.e. treatment employed to supplement another specific therapy directed toward the improvement of the associated disease, pathological condition, or disorder.

The terms “optional” and “optionally” mean that the subsequently described event, circumstance, or material may or may not occur or be present, and that the description includes instances where the event, circumstance, or material occurs or is present and instances where it does not occur or is not present.

The terms “patient” and “subject” are used interchangeably herein, and mean and include warm blooded mammals, humans and primates; avians; domestic household or farm animals, such as cats, dogs, sheep, goats, cattle, horses and pigs; laboratory animals, such as mice, rats and guinea pigs; fish; reptiles; zoo and wild animals; and the like.

The term “comprise” and variations of the term, such as “comprising” and “comprises,” means “including, but not limited to” and is not intended to exclude, for example, other additives, components, integers or steps.

The following disclosure is provided to further explain in an enabling fashion the best modes of performing one or more embodiments of the present invention. The disclosure is further offered to enhance an understanding and appreciation for the inventive principles and advantages thereof, rather than to limit in any manner the invention. The invention is defined solely by the appended claims including any amendments made during the pendency of this application and all equivalents of those claims as issued.

In overview, the present disclosure is directed to non-antigenic, resilient, bioremodelable, biocompatible annular prostheses that can be engineered into a variety of shapes and used to repair or replace a defective annulus in a mammalian heart valve. According to the invention, the annular prostheses of the invention can be readily designed and configured and, hence, employed to replace native valve annuli in the body including, without limitation, diseased or defective aortic, pulmonary, mitral, and tricuspid valves.

As indicated above, in a preferred embodiment, the annular prostheses comprise at least one elongated tubular member.

In some embodiments, the annular prosthesis comprises a biocompatible elongated tubular member.

In some embodiments, the annular prosthesis comprises a bioremodelable elongated tubular member.

In some embodiments, the tubular member comprises a substantially circular or elliptical shape.

In some embodiments, the tubular member comprises at least one planar surface, e.g. a “D” shaped tubular member.

In some embodiments, the tubular member comprises a rolled bioremodelable sheet.

In some embodiments, the tubular member comprises a rolled biocompatible sheet.

In some embodiments, the sheet comprises at least one defined edge.

In some embodiments, the sheet is circular and/or elliptical.

In some embodiments, the tubular member comprises a plurality of laminate sheets.

In some embodiments, the plurality of laminate sheets is rolled from a first edge of the laminate to form a tubular member. (See FIG. 3A)

In some embodiments, the tubular member comprises a plurality of laminate sheets having first and second opposing edges rolled and secured at linear axis (“L_{A}”) to form a tubular member. (See FIG. 4A)

In some embodiments, the laminate sheets comprise a securing means configured to retain the shape or the configuration of the tubular member including, without limitation, heat sealing, suturing, biocompatible polymer glue, fibrin glue, and platelet-fibrin glue.

In some embodiments, the tubular member comprises a plurality of biocompatible strands.

In some embodiments, the tubular member comprises a plurality of bioremodelable strands.

According to the invention, a plurality of strands can be oriented in various configurations to form a fiber construct, i.e. a bundle of strands.

Thus, in some embodiments, the fiber constructs comprise an intertwined configuration.

In some embodiments, the fiber constructs comprise a braided configuration.

In some embodiments, the fiber constructs comprise a plurality of bonded parallel strands.

In some embodiments, the fiber constructs comprise a plurality of bonded perpendicular strands.

In some embodiments, the fiber constructs comprise a plurality of loosely woven strands.

In some embodiments, the fiber constructs comprise a plurality of nonwoven strands.

In some embodiments, the fiber constructs comprise one of the aforementioned configurations having at least one strand and/or additional fiber construct wound about the outer surface of the fiber constructs.

According to the invention, the plurality of strands and/or fiber constructs can be oriented in various configurations to form a tubular member.

In some embodiments, the tubular members comprise a plurality of strands and/or fiber constructs oriented in a parallel plane.

In some embodiments, the tubular members comprise a plurality of woven planar perpendicularly intersecting strands and/or fiber constructs.

In some embodiments, the tubular members comprise a plurality of fibers forming a planar nonwoven structure having a random arrangement of strands and/or fiber constructs.

In some embodiments, the ratio of tubular member to strand and/or fiber construct size is at least 1:2, more preferably in the range of 1:2-1:1000.

In some embodiments, the tubular member comprises a surface layer.

In some embodiments, the surface layer comprises a plurality of the aforementioned strands and/or fiber constructs.

In some embodiments, the tubular member comprises a length (“L₁”) in the range of 1-500 mm. (See FIGS. 3B and 4B)

In some embodiments, the tubular member comprises a diameter (“D₁”) in the range of 1-50 mm.

In some embodiments, the tubular member comprises a tensile strength in the range of approximately 200 to 5000 KPa.

In some embodiments, the Young's modulus of the tubular member is in the range of approximately 30 to 1500 GPa.

In some embodiments, the tubular member comprises a first end and a second end.

According to the invention, the annular prostheses are formed by orienting the tubular member in a substantially circular and/or elliptical configuration and securing the first end to the second end at a junction. (See FIG. 1A)

In some embodiments, the first end and the second end are unbound.

In some embodiments, the first end and the second end comprise at least one tether therebetween.

In some embodiments, the first end and the second end comprise at a plurality of tethers therebetween.

In some embodiments, the tethers comprise bioremodelable strands and/or fiber constructs.

In some embodiments, the tethers comprise biocompatible strands and/or fiber constructs.

In some embodiments, the tethers comprise a helical configuration.

In some embodiments, the tethers comprise a length (“Z₁”) in the range of 0.1-50 mm. (see FIG. 1A)

In some embodiments, the tethers comprise a tensile strength in the range of approximately 200 to 3000 KPa.

In some embodiments, the Young's modulus of the tethers is in the range of approximately 20 to 800 KPa.

According to the invention, the tubular member can be oriented in various configurations to form an annular prostheses.

According to the invention, the annular prostheses can be configured to conform to individual physiological structural features.

In some embodiments, the annular prostheses can be sized for placement of at least 60% of the annular circumference of the valve, more preferably at least 75%.

In some embodiments, the annular prostheses are sized for the annular circumference of the valve.

In some embodiments, the annular prostheses comprise an expansion ratio of approximately 1:2:1, more preferably, at least 2:1, even more preferably 3:1 when hydrated.

As indicated above, in some embodiments of the invention, the annular prostheses comprise an ECM composition derived from a mammalian tissue source.

In a preferred embodiment of the invention, the ECM composition comprises an ECM material from a mammalian tissue source. According to the invention, the mammalian tissue sources include, without limitation, the small intestine, large intestine, stomach, lung, liver, kidney, pancreas, placenta, heart, bladder, prostate, tissue surrounding growing enamel, tissue surrounding growing bone, and any fetal tissue from any mammalian organ.

The ECM material can thus comprise, without limitation, small intestine submucosa (SIS), urinary bladder submucosa (UBS), stomach submucosa (SS), central nervous system tissue, dermal extracellular matrix, subcutaneous extracellular matrix, gastrointestinal extracellular matrix, i.e. large and small intestines, tissue surrounding growing bone, placental extracellular matrix, omentum extracellular matrix, epithelium of mesodermal origin, i.e. mesothelial tissue, cardiac extracellular matrix, e.g., pericardium and/or myocardium, kidney extracellular matrix, pancreas extracellular matrix, lung extracellular matrix, and combinations thereof.

The ECM material can also comprise collagen from mammalian sources.

In a preferred embodiment, the mammalian tissue source comprises an adolescent mammalian tissue source, i.e. an adolescent mammal, such as a piglet, which is preferably less than three (3) years of age.

The ECM can also be derived from the same or different mammalian tissue sources, as disclosed in Co-Pending application Ser. Nos. 13/033,053 and 13/033,102; which are incorporated by reference herein.

According to the invention, the ECM material can be used in whole or in part, so that, for example, an ECM material can contain just the basement membrane (or transitional epithelial layer) with the subadjacent tunica propria, the tunica submucosa, tunica muscularis, and tunica serosa. The ECM material component of the composition can contain any or all of these layers, and thus could conceivably contain only the basement membrane portion, excluding the submucosa. However, generally, and especially since the submucosa is thought to contain and support the active growth factors and other proteins necessary for in vivo tissue regeneration, the ECM or matrix composition from any given source will contain the active extracellular matrix portions that support cell development and differentiation and tissue regeneration.

In some embodiments, the ECM material comprises the submucosal layer.

In some embodiments, the ECM material comprises the epithelial basement membrane.

In some embodiments, the ECM material comprises the submucosal and the mucosal layers further comprising the muscularis mucosae therebetween.

In some embodiments, the ECM material comprises the submucosal, mucosal and muscularis layers.

In some embodiments the ECM material comprises the submucosal, mucosal, muscularis and serosa layers.

According to the invention, any of the aforementioned layers of the ECM material can be delaminated to accommodate various structures and applications.

In a preferred embodiment, the ECM Material comprises sterilized acellular ECM material.

According to the invention, the ECM can also be sterilized via applicant's proprietary novasterilis process disclosed in Co-Pending U.S. application Ser. No. 13/480,205; which is expressly incorporated by reference herein in its entirety.

In some embodiments, the ECM material is blended with an alginate to form an expandable composition having an expansion ratio of at least 5:1.

In some embodiments of the invention, the annular prostheses comprise a biocompatible polymeric composition.

According to the invention, the polymeric composition can comprise, without limitation, polycaprolactone (PCL), Artelon® (porous polyurethaneurea), polyglycolide (PGA), polylactide (PLA), poly(ε-caprolactone) (PCL), poly dioxanone (a polyether-ester), poly lactide-co-glycolide, polyamide esters, polyalkalene esters, polyvinyl esters, polyvinyl alcohol, and polyanhydrides. Natural polymeric compositions, include, without limitation, polysaccharides (e.g. starch and cellulose), proteins (e.g., gelatin, casein, silk, wool, etc.), and polyesters (e.g., polyhydroxyalkanoates).

The polymeric composition can also comprise a hydrogel composition, including, without limitation, polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide.

In some embodiments, the polymeric composition is plasma treated to accommodate hygroscopic agents.

As indicated above, in some embodiments, the polymeric composition includes at least one of the aforementioned biologically active or pharmacological agents.

In some embodiments, the polymeric composition comprises an ECM-mimicking biomaterial composition.

In some embodiments, the ECM-mimicking biomaterial composition comprises poly(glycerol sebacate) (PGS).

Applicant has found that PGS exhibits numerous beneficial properties that provide several beneficial biochemical actions or activities. The properties and beneficial actions resulting therefrom are discussed in detail below.

### PGS Physical Properties

PGS is a condensate of the non-immunogenic compositions glycerol (a simple sugar alcohol) and sebacic acid (a naturally occurring dicarboxylic acid), wherein, glycerol and sebacic acid are readily metabolized when proximate mammalian tissue. The non-immunogenic properties substantially limit the acute inflammatory responses typically associated with other “biocompatible” polymers, such as ePTFE (polytetrafluoroethylene), that are detrimental to bioremodeling and tissue regeneration.

The mechanical properties of PGS are substantially similar to that of biological tissue, wherein, the value of the Young's modulus of PGS is between that of a ligament (in KPa range) and tendon (in GPa range). The strain to failure of PGS is also similar to that of arteries and veins (i.e. over 260% elongation).

The tensile strength of the PGS is at least 0.28±0.004 MPa. The Young's modulus and elongation are at least 0.122±0.0003 and at least 237.8±0.64%, respectively. For applications requiring stronger mechanical properties and a slower biodegradation rate, PGS can be blended with poly(ε-caprolactone) PCL, i.e. a biodegradable elastomer.

### ECM Mimicking Properties/Actions

It has been found that PGS induces tissue remodeling and regeneration when administered proximate to damaged tissue, thus, mimicking the seminal regenerative properties of ECM and, hence, an ECM composition formed therefrom. The mechanism underlying this behavior is deemed to be based on the mechanical and biodegradation kinetics of the PGS. See Sant, et al., Effect of Biodegradation and de novo Matrix Synthesis on the Mechanical Properties of VIC-seeded PGS-PCL scaffolds, Acta. Biomater., vol. 9(4), pp. 5963-73 (2013).

In some embodiments of the invention, the ECM-mimicking biomaterial composition comprises PGS and at least one ECM material.

In some embodiments of the invention, the ECM-mimicking biomaterial composition comprises PGS and PCL.

In some embodiments of the invention, the ECM-mimicking biomaterial composition comprises an ECM-PGS composition, e.g. 50% ECM/50% PGS.

In some embodiments, the ECM-PGS composition further comprises PCL.

In some embodiments, the ECM-mimicking biomaterial composition comprises poly(glycerol sebacate) acrylate (PGSA), which, according to the invention, can be crosslinked and/or cured via the combination of a photoinitiator and radiation.

According to the invention, suitable photoinitiators for radiation induced crosslinking comprise, without limitation, 2-hydroxy-1-[4-hydroxyethoxy) phenyl]-2-methyl-1-propanone (D 2959, Ciba Geigy), 2,2-dimethoxy-2-phenylacetophenone, titanocenes, fluorinated diaryltitanocenes, iron arene complexes, manganese decacarbonyl, methylcyclopentadienyl manganese tricarbonyl and any organometallatic photoinitiator that produces free radicals or cations.

According to the invention, suitable radiation wavelengths for crosslinking and/or curing the ECM-mimicking biomaterial composition comprise, without limitation, visible light; particularly, radiation in the range of approximately 380-750 nm, and ultraviolet (UV) light, particularly, radiation in the range of 10-400 nm, which includes extreme UV (10-121 nm), vacuum UV (10-200 nm), hydrogen lyman α-UV (121-122 nm), Far UV (122-200 nm), Middle UV (200-300 nm), Near UV (300-400 nm), UV-C (100-280 nm), UV-B (280-315 nm) and UV-A (315-400 nm) species of UV light.

In some embodiments, the ECM-mimicking biomaterial composition comprises a co-polymer of PGSA and polyethylene glycol (PEG) diacrylate.

Preferably, the ratio of PGSA to PEG diacrylate used when developing the photocured PGSA is proportional to the physical strength of the biomaterial composition, wherein a ratio of PGSA to PEG diacrylate in the range of 95:05-50:50 comprises a Young's modulus in the range of approximately 0.5-20 MPa respectively.

According to the invention, the Young's modulus will vary based on the configuration of the annular prostheses.

In some embodiments, the annular prostheses comprise a blended plurality of ECM and/or polymeric and/or ECM-mimicking biomaterial and/or ECM/ECM-mimicking biomaterial composition sheets.

In some embodiments, the fiber constructs comprise a blended plurality of ECM and/or polymeric and/or ECM-mimicking biomaterial and/or ECM/ECM-mimicking biomaterial composition strands.

In some embodiments, the annular prostheses comprise a blended plurality of ECM and/or polymeric and/or ECM-mimicking biomaterial and/or ECM/ECM-mimicking biomaterial composition strands and/or fiber constructs.

As indicated above, in some embodiments of the invention, the sheet(s) and/or strand(s) and/or fiber construct(s) and/or annular prosthese(s) formed therefrom, include at least one additional biologically active agent or composition, i.e. an agent that induces or modulates a physiological or biological process, or cellular activity, e.g., induces proliferation, and/or growth and/or regeneration of cells and/or tissue.

Suitable biologically active agents include any of the aforementioned biologically active agents, including, without limitation, the aforementioned cells, proteins and growth factors.

In some embodiments, the sheet(s) and/or strand(s) and/or fiber construct(s) and/or annular prosthese(s) formed therefrom, include at least one pharmacological agent or composition (or drug), i.e. an agent or composition that is capable of producing a desired biological effect in vivo, e.g., stimulation or suppression of apoptosis, stimulation or suppression of an immune response, etc.

Suitable pharmacological agents and compositions include any of the aforementioned agents, including, without limitation, antibiotics, anti-viral agents, analgesics, steroidal anti-inflammatories, non-steroidal anti-inflammatories, anti-neoplastics, anti-spasmodics, modulators of cell-extracellular matrix interactions, proteins, hormones, enzymes and enzyme inhibitors, anticoagulants and/or anti-thrombic agents, DNA, RNA, modified DNA and RNA, NSAIDs, inhibitors of DNA, RNA or protein synthesis, polypeptides, oligonucleotides, polynucleotides, nucleoproteins, compounds modulating cell migration, compounds modulating proliferation and growth of cells and/or tissue, and vasodilating agents.

In some embodiments of the invention, the pharmacological agent comprises a statin, i.e. a HMG-CoA reductase inhibitor. According to the invention, suitable statins include, without limitation, atorvastatin (Lipitor®), cerivastatin, fluvastatin (Lescol®), lovastatin (Mevacor®, Altocor Altoprev®), mevastatin, pitavastatin (Livalo®, Pitava®), pravastatin (Pravachol®, Selektine®, Lipostat®), rosuvastatin (Crestor®), and simvastatin (Zocor®, Lipex®). Several actives comprising a combination of a statin and another agent, such as ezetimbe/simvastatin (Vytorin®), are also suitable.

Applicant has found that the noted statins exhibit numerous beneficial properties that provide several beneficial biochemical actions or activities. In particular, Applicant has found that when a statin is added to ECM (wherein a statin augmented ECM composition is formed) and the statin augmented ECM composition is administered to damaged tissue, the statin interacts with the cells recruited by the ECM, wherein the statin augmented ECM composition modulates inflammation of the damaged tissue by modulating several significant inflammatory processes, including restricting expression of monocyte chemoattractant protein-1 (MCP-1) and chemokine (C-C) motif ligand 2 (CCR2).

The properties and beneficial actions are discussed in detail in Applicant's Co-Pending application Ser. No. 13/328,287, filed on Dec. 16, 2011, Ser. No. 13/373,569, filed on Sep. 24, 2012 and Ser. No. 13/782,024, filed on Mar. 1, 2013; which are incorporated by reference herein in their entirety.

Additional suitable pharmacological agents and compositions that can be delivered within the scope of the invention are disclosed in Pat. Pub. Nos. 20070014874, 20070014873, 20070014872, 20070014871, 20070014870, 20070014869, and 20070014868; which are expressly incorporated by reference herein in its entirety.

According to the invention, the biologically active and pharmacological agents referenced above can comprise various forms. In some embodiments of the invention, the biologically active and pharmacological agents, e.g. simvastatin, comprise microcapsules that provide delayed delivery of the agent contained therein.

In some embodiments of the invention, the biologically active agent comprises a protein selected from the group comprising, without limitation, collagen (types I-V), proteoglycans, glycosaminoglycans (GAGs), glycoproteins, cytokines, cell-surface associated proteins, and cell adhesion molecules (CAMs).

In some embodiments, the biologically active agent provides a structural support scaffold comprising at least one layer. Suitable bioactive agents include, without limitation, elastin and ECM having additional GAG content, such as additional hyaluronic acid and/or chondroitin sulfate.

In some embodiments, the ECM composition provides a single-stage agent delivery profile, i.e. comprises a single-stage delivery vehicle, wherein a modulated dosage of an aforementioned biologically active and/or pharmacological agent is provided.

According to the invention, the term “modulated dosage” as used herein, and variants of this language generally refer to the modulation (e.g., alteration, delay, retardation, reduction, etc.) of a process involving different eluting or dispersal rates of an agent within biological tissue.

In some embodiments, the single-stage delivery vehicle comprises encapsulated particulates of a biologically active and/or pharmacological agent.

In some embodiments, the encapsulation composition comprises at least one aforementioned ECM composition.

In some embodiments, the encapsulation composition comprises at least one aforementioned polymeric composition.

In some embodiments of the invention, the encapsulation composition comprises at least one aforementioned ECM-mimicking biomaterial composition.

In some embodiments of the invention, the encapsulation composition comprises at least one aforementioned ECM/ECM-mimicking biomaterial composition.

In some embodiments, the encapsulation composition comprises an osmotic fluctuation inducing composition. According to the invention, suitable osmotic fluctuation inducing compositions include, without limitation, polyethylene glycol, alginate and dextran.

According to the invention, the term “osmotic fluctuation” as used herein, and variants of this language generally refer to the modulation of the osmotic pressure gradient across a defined barrier.

For example, as is well known in the art, alginate is capable of absorbing 200-300 times its weight in water, which substantially increases the osmotic pressure gradient of the alginate. The increased osmotic pressure gradient of the alginate results in a rapid dispersal of an agent therefrom.

In some embodiments of the invention, the ECM composition comprises a multi-stage agent delivery profile, i.e. comprises a multi-stage agent delivery vehicle, wherein a plurality of the aforementioned biologically active and/or pharmacological agents are administered via a modulated dosage. By way of example, in some embodiments, the multi-stage delivery vehicle comprises an antibiotic composition encapsulated in an alginate composition having a statin incorporated therein, which provides a tiered modulated agent delivery.

In some embodiments, the multi-stage agent delivery vehicle comprises a combination of different biologically active and/or pharmacological agents. By way of example, in some embodiments, the multi-stage delivery vehicle comprises an encapsulated growth factor concomitantly administered with an encapsulated anti-inflammatory.

In some embodiments, the multi-stage delivery vehicle comprises a plurality of different biologically active and/or pharmacological agents encapsulated in different encapsulation compositions. By way of example, in some embodiments, the multi-stage delivery vehicle comprises a growth factor encapsulated in alginate composition and a pharmacological agent encapsulated in a polyglycolide composition.

In some embodiments, the sheet(s) and/or strand(s) and/or fiber construct(s) and, hence, annular prosthese(s) formed therefrom comprise at least one coating.

Suitable coatings are disclosed in Co-Pending application Ser. No. 14/566,155, filed on Dec. 10, 2014, which is incorporated by reference herein.

In some embodiments, the coating comprises an ECM composition comprising at least one of the aforementioned ECM materials.

In some embodiments, the coating comprises a biodegradable polymeric composition comprising at least one of the aforementioned polymeric compositions.

In some embodiments, the coating comprises at least one of the aforementioned ECM-mimicking biomaterial compositions.

In some embodiments, the coating comprises at least one of the aforementioned ECM/ECM-mimicking biomaterial compositions.

In some embodiments, the coating comprises a blend of the aforementioned ECM and/or polymeric compositions and/or ECM-mimicking biomaterial compositions and/or ECM/ECM-mimicking biomaterial compositions.

In some embodiments of the invention, the annular prostheses further comprise an outer reinforcing structure, such as disclosed in Co-pending U.S. application Ser. No. 14/337,863, filed on Jul. 22, 2014, and Ser. Nos. 14/554,730, 14/554,795 and 14/554,847, filed on Nov. 26, 2014, which are incorporated by reference herein in their entirety.

According to the invention, the reinforcing structure can comprise a wound member or strand configuration, i.e. a thin strand wound around the outer surface of the tubular member, such as disclosed in Co-Pending application Ser. No. 14/337,863 or a mesh structure, such as disclosed in Co-Pending application Ser. Nos. 14/554,730, 14/554,795 and 14/554,847.

In some embodiments of the invention, the reinforcing structure comprises a mesh or woven structure.

In some embodiments of the invention, the reinforcing structure comprises one of the aforementioned ECM materials.

In some embodiments, the reinforcing structure comprises one of the aforementioned polymeric compositions.

In some embodiments of the invention, the reinforcing structure comprises one of the aforementioned ECM-mimicking biomaterial compositions.

In some embodiments of the invention, the reinforcing structure comprises one of the aforementioned ECM/ECM-mimicking biomaterial compositions.

In some embodiments of the invention, the reinforcing structure comprises a biocompatible metal, such as stainless steel and Nitinol®.

As indicated above, in some embodiments of the invention, the sheets(s) and/or strand(s) and/or fiber construct(s) and/or biomaterial composition(s) and/or coating(s) and/or reinforcing structure(s) and, hence, annular prosthese(s) formed therefrom or therewith includes at least one additional biologically active agent or composition, i.e. an agent that induces or modulates a physiological or biological process, or cellular activity, e.g., induces proliferation, and/or growth and/or regeneration of tissue.

Suitable biologically active agents include any of the aforementioned biologically active agents, including, without limitation, the aforementioned cells, proteins and growth factors.

In some embodiments, the sheets(s) and/or strand(s) and/or fiber construct(s) and/or biomaterial composition(s) and/or coating(s) and/or reinforcing structure(s) and, hence, annular prosthese(s) formed therefrom or therewith includes include at least one pharmacological agent or composition (or drug), i.e. an agent or composition that is capable of producing a desired biological effect in vivo, e.g., stimulation or suppression of apoptosis, stimulation or suppression of an immune response, etc.

According to the invention, upon deployment of an annular prosthesis proximate to damaged and/or diseased biological tissue, “modulated healing” is effectuated.

In some embodiments, when an annular prostheses of the invention is administered proximate to a diseased and/or defective valve annulus, “modulated healing” is similarly effectuated.

The term “modulated healing”, as used herein, and variants of this language generally refer to the modulation (e.g., alteration, delay, retardation, reduction, etc.) of a process involving different cascades or sequences of naturally occurring tissue repair in response to localized tissue damage or injury, substantially reducing their inflammatory effect. Modulated healing, as used herein, includes many different biologic processes, including epithelial growth, fibrin deposition, platelet activation and attachment, inhibition, proliferation and/or differentiation, connective fibrous tissue production and function, angiogenesis, and several stages of acute and/or chronic inflammation, and their interplay with each other.

For example, in some embodiments, the annular prostheses of the invention are specifically formulated (or designed) to alter, delay, retard, reduce, and/or detain one or more of the phases associated with healing of damaged tissue, including, but not limited to, the inflammatory phase (e.g., platelet or fibrin deposition), and the proliferative phase when in contact with biological tissue.

In some embodiments, “modulated healing” refers to the ability of sheet(s) and/or strand(s) and/or fiber construct(s) and, hence, annular prosthese(s) formed therefrom to restrict the expression of inflammatory components. By way of example, according to the invention, when a sheet, strand, and/or fiber construct, and, hence, annular prosthesis formed therefrom comprises a statin augmented ECM composition, i.e. a composition comprising an ECM and an exogenously added statin, is disposed proximate damaged biological tissue, the sheet, strand and/or fiber construct and/or annular prosthesis restricts the expression of monocytes chemoattractant protein 1 (MCP-1) and chemokine (C-C) motif ligand 2 (CCR2).

In some embodiments, “modulated healing” means and includes the ability of sheet(s) and/or strand(s) and/or fiber construct(s) and, hence, annular prosthese(s) formed therefrom to alter a substantial inflammatory phase (e.g., platelet or fibrin deposition) at the beginning of the tissue healing process. As used herein, the phrases “alter a substantial inflammatory phase” refers to the ability of an annular prosthesis to substantially reduce the inflammatory response at an injury site when in contact with biological tissue.

In such an instance, a minor amount of inflammation may ensue in response to tissue injury, but this level of inflammation response, e.g., platelet and/or fibrin deposition, is substantially reduced when compared to inflammation that takes place in the absence of an annular prostheses of the invention.

For example, several sheet(s) and/or strand(s) and/or fiber construct(s) and, hence, annular prosthese(s) discussed herein have been shown experimentally to delay or alter the inflammatory response associated with damaged tissue, as well as excessive formation of connective fibrous tissue following tissue damage or injury. The annular prostheses have also been shown experimentally to delay or reduce fibrin deposition and platelet attachment to a blood contact surface following tissue damage.

The term, “modulated healing” also refers to the ability of sheet(s) and/or strand(s) and/or fiber construct(s) and, hence, annular prosthese(s) formed therefrom of the invention to induce host tissue and/or cell proliferation, bioremodeling, including neovascularization, e.g., vasculogenesis, angiogenesis, and intussusception, and regeneration of tissue structures with site-specific structural and functional properties.

Thus, in some embodiments, the term “modulated healing” also refers to the ability of sheet(s) and/or strand(s) and/or fiber construct(s) and, hence, annular prosthese(s) formed therefrom to modulate inflammation and/or induce host tissue and/or cell proliferation and remodeling. Again, by way of example, according to the invention, when sheet(s) and/or strand(s) and/or fiber construct(s) and, hence, annular prosthese(s) formed therefrom comprise a statin augmented ECM composition, i.e. a composition comprising an ECM and an exogenously added statin, is disposed proximate damaged biological tissue, the statin interacts with the cells recruited by the ECM, wherein sheet(s) and/or strand(s) and/or fiber construct(s) and, hence, annular prosthese(s) formed therefrom modulate inflammation by, among other actions, restricting expression of monocyte chemoattractant protein-1 (MCP-1) and chemokine (C-C) motif ligand (CCR2) and induces tissue and/or cell proliferation, bioremodeling and regeneration of tissue structures with site specific structural and functional properties.

By way of a further example, according to the invention, when sheet(s) and/or strand(s) and/or fiber construct(s) and, hence, annular prosthese(s) formed therefrom comprise a growth factor augmented ECM composition, i.e. a composition comprising an ECM and an exogenously added growth factor, e.g. TGF-β, is disposed proximate damaged biological tissue, the growth factor, similarly interacts with the ECM and cells recruited by the ECM, wherein the sheet(s) and/or strand(s), and/or fiber construct(s) and, hence, annular prosthese(s) formed therefrom modulate inflammation and induces tissue and/or cell proliferation, bioremodeling and regeneration of tissue.

In some embodiments, the term “modulated healing” also refers to the ability of sheets) and/or strand(s) and/or fiber construct(s) and, hence, annular prosthese(s) formed therefrom to modulate acute inflammation and/or temporarily induce an inflammatory response. By way of example, according to the invention, when a sheet and/or strand and/or fiber construct and/or annular prosthesis comprises a thrombospondin (TSP) augmented ECM composition, i.e. a composition comprising an ECM and an exogenously added TSP, is disposed proximate damaged biological tissue, the TSP, similarly interacts with the ECM and cells recruited by the ECM, wherein the sheet(s) and/or strand(s) and/or fiber construct(s) and/or annular prostheses formed therefrom modulate acute inflammation and abate poor valve leaflet apposition resulting from native annulus dilation, thus, providing a foundation for tissue and/or cell proliferation, bioremodeling and regeneration of tissue.

In some embodiments, when an annular prostheses is in contact with biological tissue modulated healing is effectuated through the structural features of an annular prostheses. The structural features provide the spatial temporal and mechanical cues to modulate cell polarity and alignment. The structural features further modulate cell proliferation, migration and differentiation thus modulating the healing process.

In some embodiments, the annular prostheses comprise an anisotropic strand and/or fiber construct structure providing spatial temporal and mechanical cues.

According to the invention, the annular prostheses of the invention can be anchored proximate to damaged and/or diseased tissue by various conventional means.

In some embodiments of the invention, the annular prostheses of the invention further include at least one anchoring mechanism that is configured to position the valves proximate cardiovascular tissue, and maintain contact therewith for a pre-determined anchor support time period. According to the invention, the anchoring mechanisms can comprise various forms and materials.

As defined above and discussed in detail in application Ser. No. 13/782,024, the terms “anchoring mechanism” and “anchor”, as used in connection with some embodiments of annular prostheses of the invention mean a structure that is configured and employed to temporarily position and support an annular prosthesis of the invention proximate host tissue of a valve annulus.

In some embodiments, the anchoring mechanisms position the annular prostheses proximate annulus of a valve, and maintain contact therewith for a predetermined temporary anchor support period of time within the process of tissue regeneration.

In some embodiments, the annular prostheses is positioned and supported proximate the host tissue of a valve annulus via sutures.

In some embodiments, the sutures comprise an aforementioned biocompatible polymer.

In some embodiments, the sutures comprise a biodegradable composition.

Suitable biodegradable compositions include, without limitation, RESOMER® polymers, i.e. poly(lactic-co-glycolic acid) (PLGA) and poly(lactic acid) (PLA).

In some embodiments, the sutures comprise an aforementioned ECM-mimicking biomaterial composition.

According to the invention, various conventional methods can be employed to form and/or extrude the aforementioned strands of the invention, including, without limitation, break spinning, open-end spinning, melt spinning, dry spinning, wet spinning, coaxial electrospinning, needleless electrospinning, and Forcespinning®.

Referring now to FIGS. 1-3, there are shown several embodiments of annular prostheses 20a, 20b and 20c respectively of the invention comprising a plurality of equidistant sutures 15 having interval lengths S₁ in the range of 1-50 mm. In the illustrated embodiment, annular prosthesis 20a, 20b and 20c can comprise an inner diameter or circumference (“C₁”) in the range of 1-100 mm and a tubular diameter (“D₁”) in the range of 1-50 mm.

As indicated above, annular prosthesis 20a, 20b and 20c can comprise various dimensions, e.g., length, circumference, etc., to accommodate various structures and applications.

As illustrated in FIG. 1A, annular prosthesis 20a can comprise a plurality of tethers 17 having a length Z₁ in the range of 0.1-50 mm secured to a first end 40a and a second end 40b of the annular prosthesis 20.

In some embodiments, the tethers 17 are configured to provide a modulated fitment upon administration of the annular prosthesis 20a to damaged tissue to dimension and configure to the unique annuli morphology of each application.

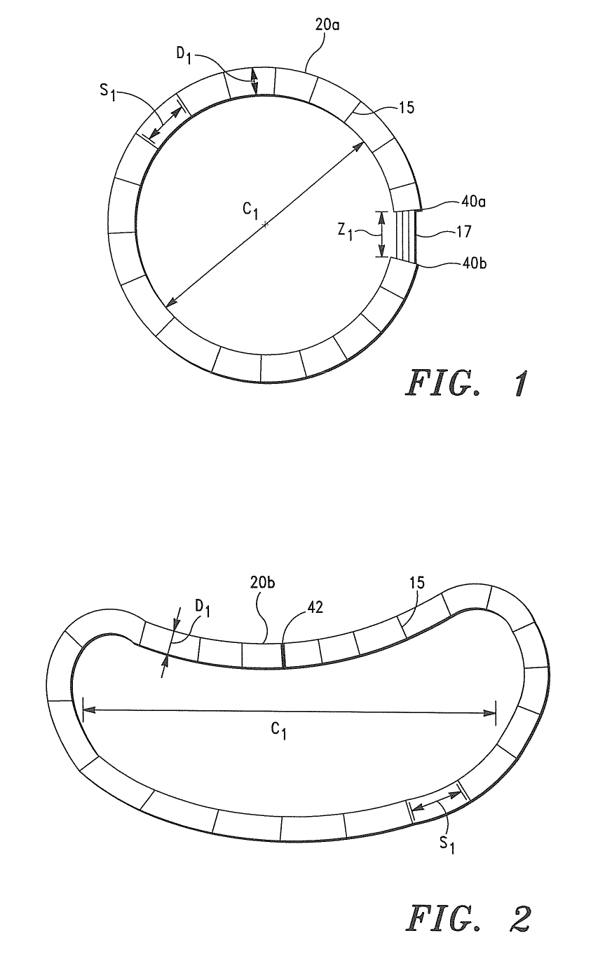

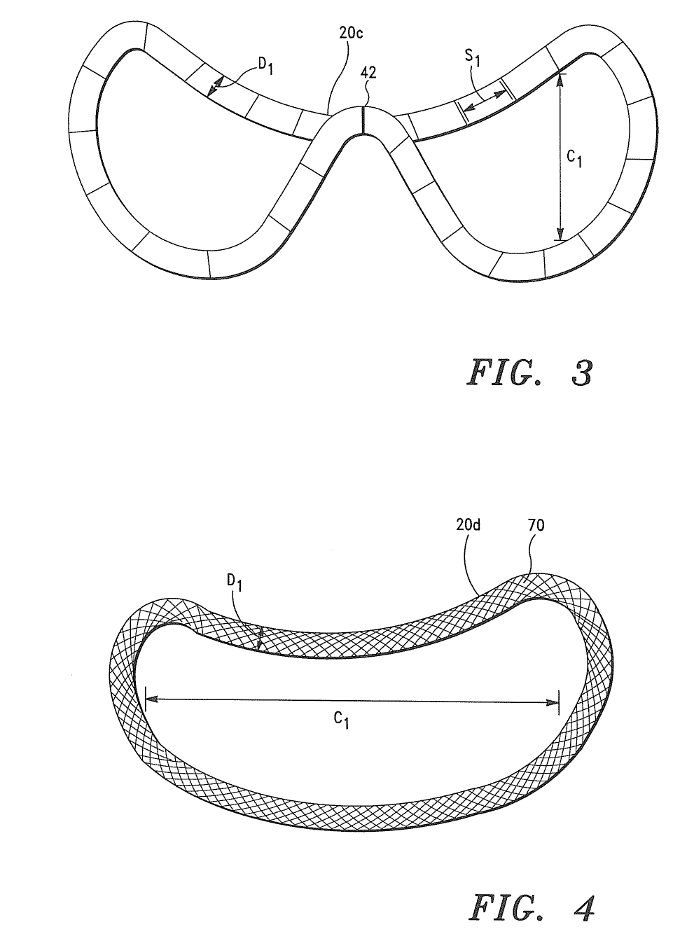

As illustrated in FIG. 2, there is shown one embodiment of an annular prosthesis 20b configured to repair and/or replace a defective bicuspid or tricuspid valve annulus having first end 40a and second end 40b bound to form junction 42.

As further illustrated in FIG. 2, the annular prostheses 20b comprise a ring dimensioned and configured to provide at least two opposing arcs configured to align with the annuli morphology of a bicuspid and/or tricuspid valve.

As illustrated in FIG. 3, there is shown another embodiment of annular prosthesis 20c configured to repair and/or replace a defective aortic or pulmonary valve annulus.

As further illustrated in FIG. 3, the annular prostheses 20c comprise a ring dimensioned and configured to provide at least three opposing arcs configured to align with the annuli morphology of an aortic and/or pulmonary valve.

Referring now to FIG. 4, there is shown another embodiment of an annular prosthesis 20d comprising a plurality of strands 70 having an interwoven configuration.

According to the invention, the annular prostheses 20d can also comprise a plurality of the aforementioned fiber constructs.

According to the invention, the annular prostheses 20d can comprise a combination of strands 70 and fiber constructs.

According to the invention, the annular prosthesis 20d can similarly comprise various dimensions to accommodate various structures and application.

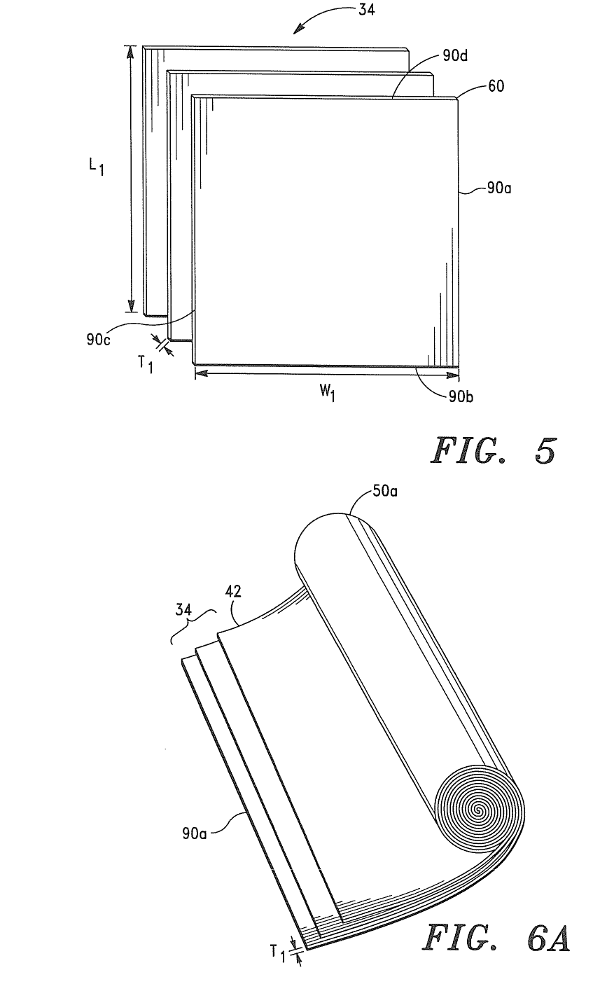

Referring now to FIG. 5, there is shown one embodiment of a laminate 34 formed from a plurality of sheets 60 comprising a first edge 90a, a second edge 90b, a third edge 90c, and a fourth edge 90d, wherein laminate 34 is configured to provide the various embodiments of the annular prostheses 20a, 20b and 20c as shown in FIGS. 1-3.

In the illustrated embodiment, the sheets 60 comprise a width (“W₁”), a thickness (“T₁”) and a length (“L₁”).

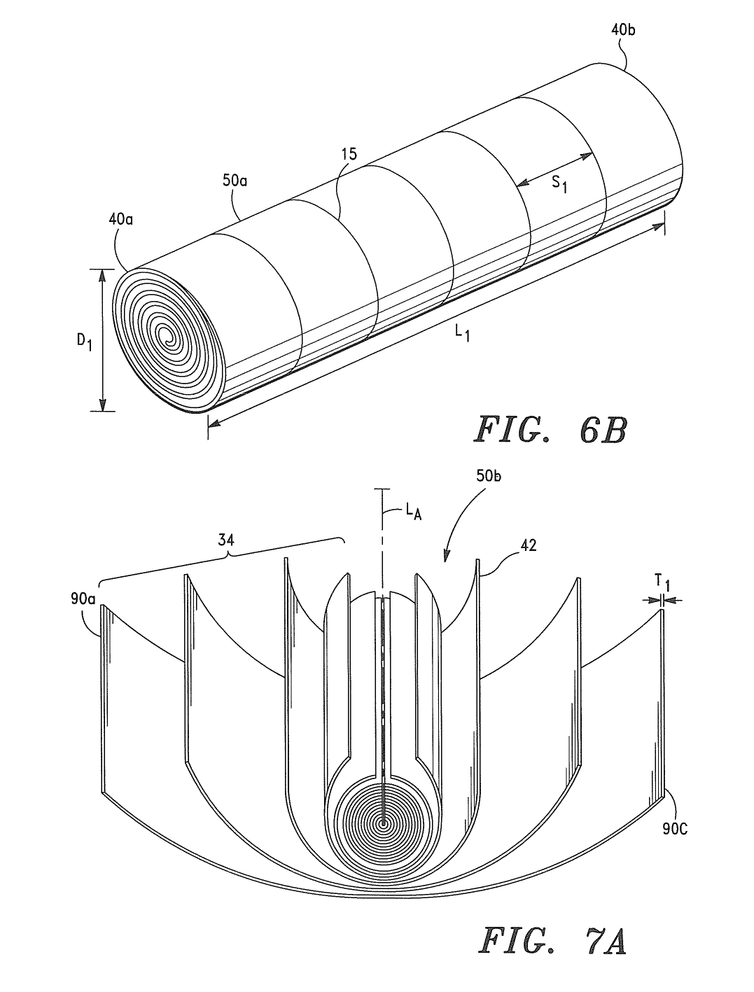

Now referring to FIGS. 6A and 6B, there is shown one embodiment of a tubular member 50a formed from a laminate 34 rolled from first edge 90a until third edge 90c contacts the surface of the tubular member 50a. According to the invention, the tubular member 50a is conformed and subsequently bound by first end 40a and second end 40b via tethers 17 to form the annular prostheses 20a, as shown in FIG. 1.

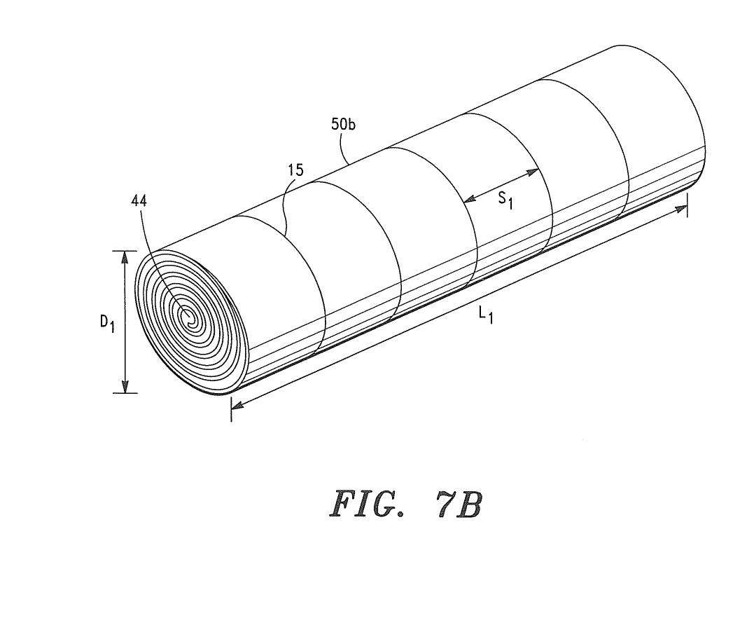

Now referring to FIGS. 7A and 7B, there is shown one embodiment of a tubular member 50b formed from a laminate 34 having first edge 90a and third edge 90c rolled, and bound at connection point 44 aligned with linear axis L_{A}. According to the invention, the tubular member 50a is conformed and subsequently bound by ends 40a, 40b via tethers 17 to form the annular prostheses 20a, as shown in FIG. 1.

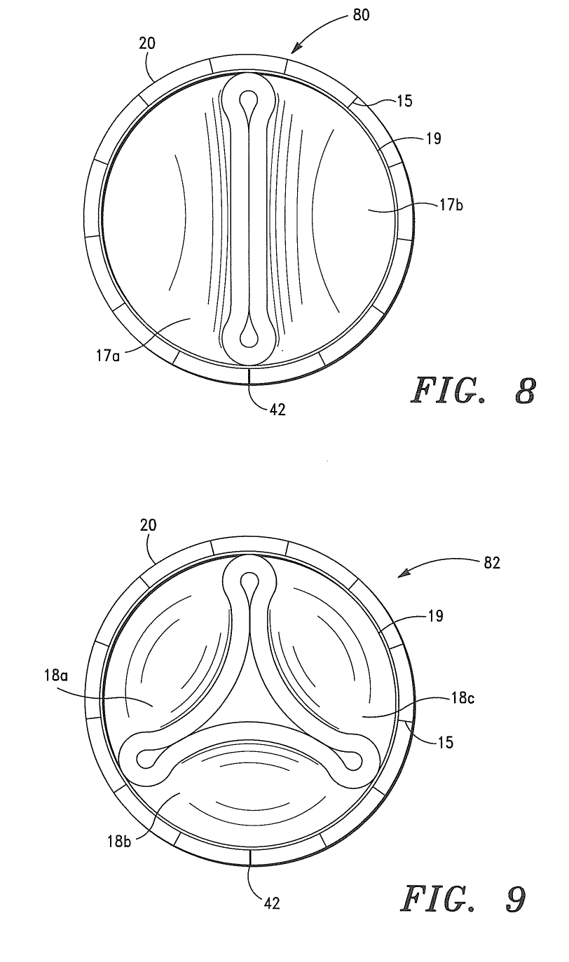

Now referring to FIG. 8, there is shown a depiction of a bicuspid valve 80 having valve leaflets 17a, 17b and a defective annulus 19. As illustrated in FIG. 5, the annular prosthesis 20b as shown in FIG. 2, is configured to repair and/or replace the defective annulus 19, wherein the annular prostheses 20b is disposed proximate to bicuspid valve 80.

Now referring to FIG. 9, there is shown a depiction of a tricuspid valve 82 having valve leaflets 18a, 18b, 18c and a defective annulus 19. As illustrated in FIG. 6, the annular prosthesis 20b as shown in FIG. 2, is configured to repair and/or replace the defective annulus 19, wherein the annular prostheses 20c is disposed proximate tricuspid valve 82.

### EXAMPLES

The following examples are provided to enable those skilled in the art to more clearly understand and practice the present invention. They should not be considered as limiting the scope of the invention, but merely as being illustrated as representative thereof.

### Example 1

An ECM patch (i.e. matrix) comprising small intestine submucosa (SIS) and 1 mg/ml of a statin, i.e. cerivastatin, was surgically applied to the myocardium of two canines. The ECM patches remained attached to the myocardium of the canines until they were sacrificed at 2 and 24 hours, respectively.

Cardiac tissue samples were collected immediately after the canines were sacrificed. The cardiac tissue samples were then subjected to mRNA extraction and quantification via established protocols.

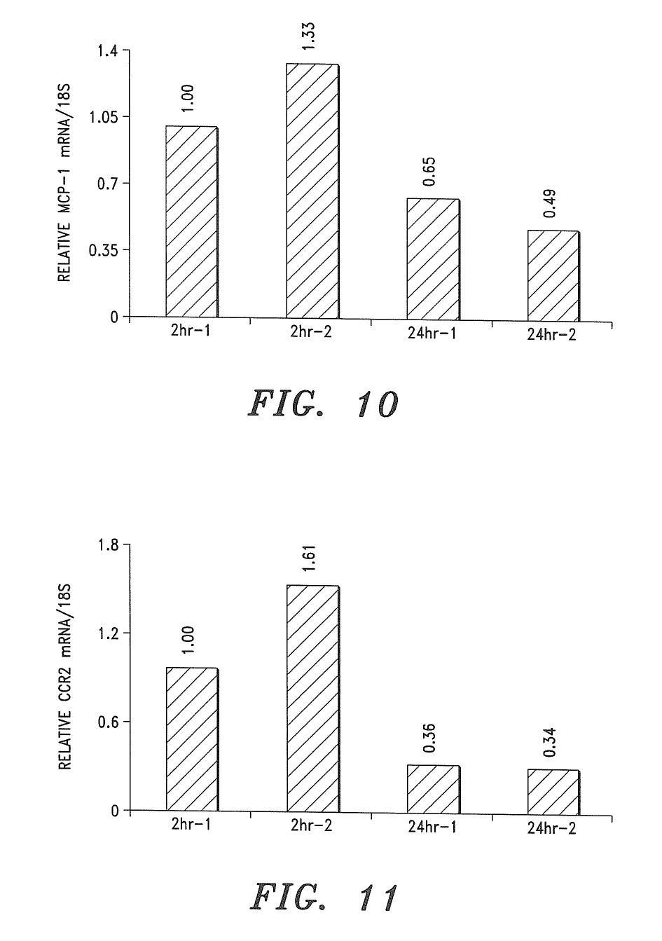

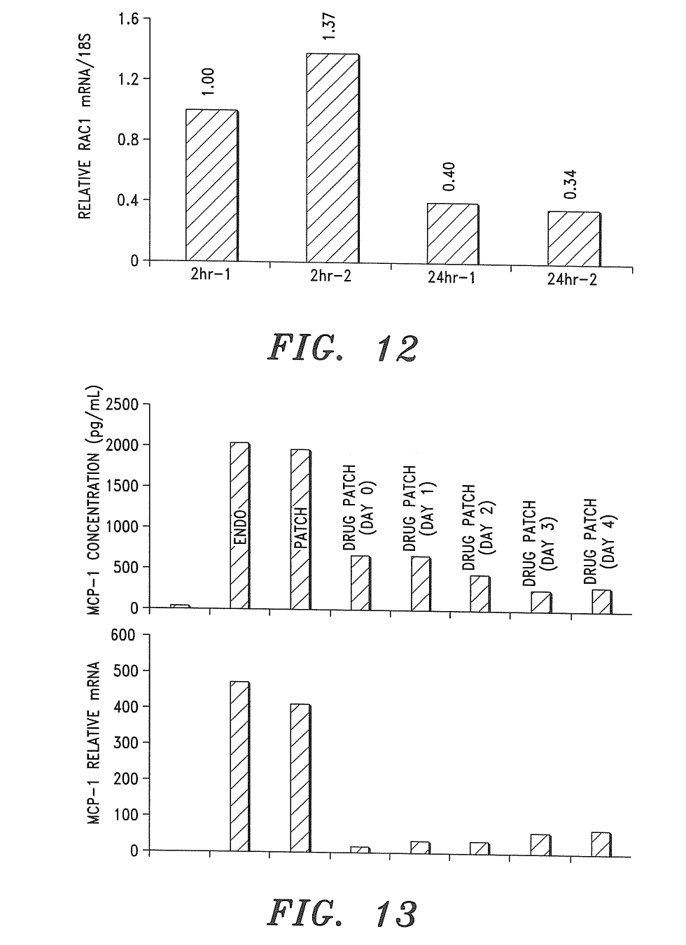

The measured mRNA levels from the cardiac tissue samples, which are shown in FIGS. 8-10, reflect substantially reduced MCP-1 and CCR2 expression at a 24 hour time point compared to the MCP-1 and CCR2 expression at a 2 hour time point. The mRNA levels thus reflect a consistent and highly effective anti-inflammatory effect over time in vivo, when a statin augmented ECM is administered to biological tissue.

The canine model experiment was further reinforced by an additional in vitro study, wherein MCP-1 expression of THP-1 cells (a human monocytic cell line) in the presence of a statin augmented ECM was analyzed. As reflected in FIG. 13, the statin augmented ECM induced substantially lower MCP-1 expression when compared to a positive control.

The example thus confirms that when a statin augmented ECM and, hence, sheet, strand, and/or fiber construct, and, hence, annular prosthesis formed therefrom, is administered to damaged tissue, the strand and/or fiber construct and, hence, annular prosthesis formed therefrom modulates several significant inflammation processes, including inhibiting generation of MCP-1 and CCR2.

As will readily be appreciated by one having ordinary skill in the art, the present invention provides numerous advantages compared to prior art annuloplasty rings. Among the advantages are the following:
The provision of annular prostheses that can be readily employed to selectively replace diseased and/or defective aortic, pulmonary, mitral and tricuspid annuli.
The provision of annular prostheses that substantially reduce or eliminate intimal hyperplasia after intervention in a vessel and the harsh biological responses associated with conventional polymeric and metal prostheses.
The provision of annular prostheses that induce host tissue and/or cell proliferation, bioremodeling and regeneration of new tissue and tissue structures with site-specific structural and functional properties.
The provision of annular prostheses that are capable of administering a pharmacological agent to host tissue and, thereby produce a desired biological and/or therapeutic effect.
The provision of annular prostheses that include anchoring mechanisms, which temporarily position the valves proximate cardiovascular tissue for a pre-determined period of time.
The provision of annular prostheses that exhibit optimum mechanical compatibility with vascular structures.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. An annular prosthesis for repairing a defective cardiovascular valve annulus, comprising:
an elongated tubular member comprising an extracellular matrix (ECM) composition, said tubular member comprising an outer surface, said ECM composition comprising acellular ECM from a mammalian tissue source,
said tubular member being configured to induce modulated healing when disposed proximate cardiovascular tissue, said modulated healing comprising modulation of inflammation and induced bioremodeling and regeneration of tissue structures with site-specific structural and functional properties.

2. The annular prosthesis of claim 1, wherein said tissue source is selected from the group consisting of the small intestine, large intestine, stomach, lung, liver, kidney, pancreas, placenta, heart, bladder, prostate, tissue surrounding growing enamel, tissue surrounding growing bone, and fetal tissue from a mammalian organ.

3. The annular prosthesis of claim 2, wherein said tissue source comprises an adolescent mammalian tissue source.

4. The annular prosthesis of claim 1, wherein said ECM composition comprises at least one supplemental biologically active agent.

5. The annular prosthesis of claim 4, wherein said supplemental biologically active agent comprises a cell selected from the group consisting of an embryonic stem cell, mesenchymal stem cell, hematopoietic stem cell, bone marrow stem cell and, bone marrow-derived progenitor cell and myosatellite progenitor cell.

6. The annular prosthesis of claim 4, wherein said supplemental biologically active agent comprises a growth factor selected from the group consisting of transforming growth factor alpha (TGF-α), transforming growth factor beta (TGF-β), fibroblast growth factor-2 (FGF-2), basic fibroblast growth factor (bFGF), vascular epithelial growth factor (VEGF), and insulin-like growth factor (IGF).

7. The annular prosthesis of claim 4, wherein said annular prosthesis comprises a multi-stage delivery vehicle.

8. The annular prosthesis of claim 1, wherein said ECM composition comprises at least one pharmacological agent.

9. The annular prosthesis of claim 8, wherein said pharmacological agent comprises an anti-inflammatory selected from the group consisting of steroidal anti-inflammatories and non-steroidal anti-inflammatories.

10. An annular prosthesis for repairing a defective cardiovascular valve annulus, comprising:
an elongated tubular member comprising an ECM-mimicking biomaterial composition, said tubular member comprising an outer surface,
said tubular member being configured to induce modulated healing when disposed proximate cardiovascular tissue, said modulated healing comprising modulation of inflammation and induced bioremodeling and regeneration of tissue structures with site-specific structural and functional properties.

11. The annular prosthesis of claim 10, wherein said ECM-mimicking biomaterial composition comprises poly(glycerol sebacate) (PGS).

12. The annular prosthesis of claim 10, wherein said ECM-mimicking biomaterial composition comprises at least one supplemental biologically active agent.

13. The annular prosthesis of claim 12, wherein said supplemental biologically active agent comprises a cell selected from the group consisting of an embryonic stem cell, mesenchymal stem cell, hematopoietic stem cell, bone marrow stem cell and, bone marrow-derived progenitor cell and myosatellite progenitor cell.

14. The annular prosthesis of claim 12, wherein said supplemental biologically active agent comprises a growth factor selected from the group consisting of transforming growth factor alpha (TGF-α), transforming growth factor beta (TGF-β), fibroblast growth factor-2 (FGF-2), basic fibroblast growth factor (bFGF), vascular epithelial growth factor (VEGF), and insulin-like growth factor (IGF).

15. The annular prosthesis of claim 12, wherein said annular prosthesis comprises a multi-stage delivery vehicle.

16. The annular prosthesis of claim 10, wherein said annular prosthesis further comprises an outer reinforcing structure, said outer reinforcing structure being disposed proximate said tubular member outer surface.

17. The annular prosthesis of claim 16, wherein said outer reinforcing structure comprises a biocompatible polymeric composition.

18. The annular prosthesis of claim 17, wherein said biocompatible polymeric composition comprises a biocompatible polymer selected from the group consisting of polycaprolactone (PCL), Artelon® (porous polyurethaneurea), polyglycolide (PGA), polylactide (PLA), poly(ε-caprolactone) (PCL), poly dioxanone (a polyether-ester), poly lactide-co-glycolide, polyamide esters, polyalkalene esters, polyvinyl esters, polyvinyl alcohol, and polyanhydrides.

18. The annular prosthesis of claim 17, wherein said biocompatible polymeric composition comprises a hydrogel composition.

19. The annular prosthesis of claim 18, wherein said hydrogel composition comprises a hydrogel selected from the group consisting of polyurethane, poly(ethylene glycol), poly(propylene glycol), poly(vinylpyrrolidone), xanthan, methyl cellulose, carboxymethyl cellulose, alginate, hyaluronan, poly(acrylic acid), polyvinyl alcohol, acrylic acid, hydroxypropyl methyl cellulose, methacrylic acid, αβ-glycerophosphate, κ-carrageenan, 2-acrylamido-2-methylpropanesulfonic acid, and β-hairpin peptide.
